(19) United States
(12) Patent Application Publication
(10) Pub. No.: US 2010/0311862 A1
(43) Pub. Date: Dec. 9, 2010

(54) RESTORING MATERIALS CONTAINING NANOCRYSTALLINE EARTH ALKALINE FILLERS

(76) Inventors: Juergen Engelbrecht, Hamburg (DE); Thomas Panther, Hamburg (DE)

Correspondence Address: EDWARDS ANGELL PALMER & DODGE LLP, P.O. BOX 55874, BOSTON, MA, 02205, US

(21) Appl. No.: 12/084,844

(22) PCT Filed: Nov. 7, 2006
(86) PCT No.: PCT/EP2006/010658

§ 371 Date: Aug. 16, 2010

(30) Foreign Application Priority Data
Nov. 10, 2005 (DE) 10 2005 053 705.7

(51) Int. Cl. A61K 6/08

(52) U.S. Cl. 523/116

(57) ABSTRACT

The invention describes restoration materials containing nano-crystalline alkaline earth fillers for use on the tooth substance, which are distinguished by high aesthetics, a high degree of hardness, excellent transparency, good surface-polish-ability, high strength and by the ability to release ions into a biological environment.

The invention describes restoration materials containing nano-crystalline alkaline earth fillers for use on the tooth substance, which materials are distinguished by high aesthetics, a high degree of hardness, excellent transparency, good surface-polishability, high strength and by the ability to release ions into a biological environment.

### PRIOR ART

There is a general desire for curable restorative dental materials that exhibit advantageous bioactive properties and are able, for example, to release fluoride, calcium or hydroxyl ions. Those ions have the advantage that they have positive effects in respect of greater remineralisation and provide the basis for a cariostatic action.

A large number of restorative dental materials are known to have ion-yielding properties, such as, for example, glass ionomer cements, resin-modified variants thereof and compomer filling materials (for example A. D. Wilson, J. W. McLean, Glass Ionomer Cement, Quintessence Publishers, Chicago, 1988).

Restorative materials of those kinds exhibit a high level of ion release, such as of calcium and fluoride.

Disadvantageous aspects, however, are insufficient resistance to acid and, in the long term, insufficient mechanical strength.

Other approaches to solving those problems are the use of special strongly ion-yielding glasses which are used as composite fillers in the form of extremely fine powders on a μm scale, which exhibit a high fluoride yield on contact with water and which allow aesthetic restorations with good transparency (DE 197 57 645).

Glasses used therein having an “invert glass structure” contain fluorides in amounts far higher than normal glasses, the fluorides apparently being present in that glass structure, for example, in the form of CaF₂.

EP 0 002 831 describes opaque dental filling materials containing finely divided calcium fluoride as white pigment. Calcium fluoride is lauded as being a pigment that imparts opacity but nevertheless does not prevent photocuring. The particle size of the calcium fluoride lies in the μm range. Nothing is said about any noticeable release of fluoride.

EP 1 139 995 describes the preparation of sparingly soluble calcium salts, inter alia calcium fluoride, in a particle size in the nanometre range and the use thereof in dental care preparations. Calcium fluoride is described therein as being useful for strengthening the enamel and for preventing caries, especially when it is used in extremely finely divided form.

The problem underlying the invention was to find curable restorative dental materials that exhibit advantageous bioactive properties and are able, for example, to release fluoride ions, calcium ions or other alkaline earth ions—but without their having a substantial opacifying effect—, that can have aesthetic optical properties and that are able to withstand long-term mechanical and chemical loading in the mouth.

The problem has been solved according to the invention by dental restoration materials based on a crosslinkable resin system and/or a curing acid/base cement system, which contains nanostructured alkaline earth fluoride, preferably strontium fluoride, especially calcium fluoride, the particle size of the alkaline earth fluoride lying predominantly in the range of from 2 to 200 nm.

### DESCRIPTION OF THE INVENTION

In line with the problem, it has been found according to the invention that the use of nanocrystalline alkaline earth fillers (optionally with additions of other fillers, such as, for example, glasses or microfine silica) in curable restoration materials either based on curable resin systems or cement systems customarily used in dental materials or combinations thereof results in a desirable tooth-like transparency and an ability to withstand high mechanical loading. In addition, those materials exhibit the desired ability to release ions and to absorb ions.

In the context of the invention, curable restorative dental materials should be understood as referring to those materials which are employed for dental fillings, inlays or onlays, fixing cements, veneer materials for crowns and bridges, materials for artificial teeth, dentine and enamel bondings, lining materials, stump build-up materials, root-filling materials or other curable materials for prosthetic, conservative and preventive dentistry.

The dental materials of the present invention are able to yield fluoride ions and alkaline earth ions into their biological environment and fulfil the aesthetic and physical properties required of dental materials.

The curable dental materials consist of a curable matrix (on the one hand as crosslinkable resins and/or on the other hand as acid/base cements) and alkaline earth fluorides and if required other fillers and if required other additives.

An important group of matrices from the group of crosslinkable resins that can be used successfully includes polymerisable, ethylenically unsaturated monomers, preferably those having acrylic and/or methacrylic groups.

In particular, they include inter alia esters of cyanoacrylic acid, (meth)acrylic acid, urethane (meth)acrylic acid, crotonic acid, cinnamic acid, sorbic acid, maleic acid and itaconic acid with mono- or poly-hydric alcohols; (meth)acrylamides such as, for example, N-isobutylacrylamide; vinyl esters of carboxylic acids such as, for example, vinyl acetate; vinyl ethers such as, for example, butyl vinyl ether; mono-N-vinyl compounds such as N-vinylpyrrolidone; and styrene and derivatives thereof. Special preference is given to the mono- and poly-functional (meth)-acrylic acid esters and urethane (meth)acrylic acid esters listed below:

(a) Monofunctional (meth)acrylates

methyl (meth)acrylate, n- or iso-propyl (meth)acrylate, n-, iso- or tert-butyl (meth)acrylate and 2-hydroxyethyl (meth)acrylate.

(b) Difunctional (meth)acrylates

Compounds of the general formula:

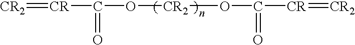

wherein R is hydrogen or methyl and n is a positive whole number from 3 to 20, such as,
for example, the di(meth)acrylates of propanediol, butanediol, hexanediol, octanediol, nonanediol, decanediol and eicosanediol,

compounds of the general formula:

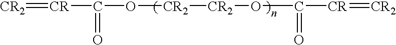

wherein R is hydrogen or methyl and n is a positive whole number from 1 to 14, such as,
for example, the di(meth)acrylates of ethylene glycol, diethylene glycol, triethylene glycol, tetraethylene glycol, dodecaethylene glycol, tetradecaethylene glycol, propylene glycol, dipropyl glycol and tetradecapropylene glycol; and glycerol di(meth)acrylate, 2,2′-bis(4(3′-methacryloyloxy-2′-hydroxypropoxy)phenyl]propane) or bis-GMA, biphenol-A-dimethacrylate, neopentyl glycol di(meth)acrylate, 2,2′-di(4-meth-acryloxypolyethoxyphenyl)propane having from 2 to 10 ethoxy groups per molecule and 1,2-bis(3-methacryloxy-2-hydroxy-propoxy) butane.
(c) Trifunctional or polyfunctional (meth)acrylates

trimethylolpropane tri(meth)acrylates and

pentaerythritol tetra(meth)acrylate.

(d) Urethane (meth)acrylates, which are usually obtained by reacting 2 mol of hydroxyl-group-containing (meth)acrylate monomer with one mol of diisocyanate.
e) Siloxane-based mono-, di- or multi-acrylates
Siloxane-based mono-, di- or multi-acrylates are also importance in this context.

The mentioned monomers are used either alone or in the form of a mixture of several monomers.

Monomers that are very especially advantageously used in the restoration material according to the invention include especially 2,2-bis-4(3-methacryloxy-2-hydroxypropoxy)-phenylpropane (bis-GMA), 3,6-dioxaoctamethylene dimethacrylate (TEDMA) and/or 7,7,9-trimethyl-4,13-dioxo-3,14-dioxa-5,12-diazahexadecane-1,16-dioxy-dimethacrylate (UDMA).

The monomers may also carry basic groups, such as, for example, amines, or acid groups, as described, for example, in U.S. Pat. No. 4,806,381.

According to the nature of the catalyst used, the material can be hot-polymerisable, cold-polymerisable and/or photopolymerisable. As catalysts for hot-polymerisation there may be used the known peroxides, such as dibenzoyl peroxide, dilauroyl peroxide, tert-butyl peroctoate or tert-butyl perbenzoate, but azo-bis(isobutyroethyl ester), benzpinacol and 2,2′-dimethyl-benzpinacol are also suitable.

As catalysts for photopolymerisation there may be used, for example, benzophenone and derivatives thereof, and also benzoin and derivatives thereof. Further preferred photosensitisers are α-diketones such as 9,10-phenanthrenequinone, diacetyl, furil, anisil, 4,4′-dichlorobenzil and 4,4′-dialkoxybenzil. Special preference is given to the use of camphorquinone. The use of photosensitisers together with a reducing agent is preferred. Examples of reducing agents are amines, such as cyanoethyl-methylaniline, dimethylaminoethyl methacrylate, triethylamine, triethanolamine, N,N-dimethylaniline, N-methyldiphenylamine, N,N-dimethyl-sym.-xylidine and N,N-3,5-tetramethylaniline and 4-dimethylaminobenzoic acid ethyl ester.

As catalysts for cold-polymerisation there are used systems that yield free radicals, for example benzoyl or lauroyl peroxide together with amines such as N,N-dimethyl-sym.-xylidine or N,N-dimenthyl-p-toluidine. It is also possible to use dual-curing systems for catalysis, for example photoinitiators with amines and peroxides. As photocatalysts there also come into consideration mixtures of UV-light-curing catalysts and catalysts curing in the range of visible light.

The amount of such catalysts in the dental material is usually from 0.01 to 5% by weight.

Another group of resin matrices that can successfully be used includes ring-opening monomers that undergo polymerisation or polyaddition with ring-opening.

Examples include organic compounds having, for example, an oxirane ring, that is to say

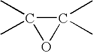

which is polymerisable by ring-opening. Such materials are generally referred to as epoxides; they include monomeric epoxy compounds and also epoxides of the polymeric type and can be aliphatic, cycloaliphatic, aromatic or heterocyclic compounds. Those materials generally have on average at least one polymerisable epoxy group per molecule, preferably at least about 1.5 polymerisable epoxy groups per molecule. The polymeric epoxides comprise linear polymers having terminal epoxy groups (for example a diglycidyl ether of a polyoxyalkylene glycol), polymers having oxirane units in the molecular structure (e.g. polybutadiene-polyepoxide) and polymers having epoxy groups attached to the structure (e.g. a glycidyl methacrylate polymer or copolymer). Those epoxides can be pure compounds or mixtures containing one, two or more epoxy groups per molecule.

Suitable epoxy-containing materials include materials containing cyclohexene oxide groups, such as, for example, the epoxycyclo-hexanecarboxylates; typical examples are 3,4-epoxycyclohexyl-methyl-3,4-epoxycyclohexanecarboxylate, 3,4-epoxy-2-methylcyclo-hexylmethyl-3,4-epoxy-2-methylcyclohexanecarboxylate and also bis(3,4-epoxy-6-methyl-cyclohexylmethyl) adipate. For a more detailed list of suitable epoxides of this kind reference is made to U.S. Pat. No. 3,117,099, which is incorporated herein by reference.

Further suitable epoxy resins are silicones having epoxide functionality, especially cyclohexylepoxide groups, especially those having a silicone underlying framework. Examples are UV 9300, UV 9315, UV 9400 and UV 9425, which are all available from GE Bayer Silicones.

Very especially suitable and especially biocompatible are monomers having epoxy functions and organic underlying frame-works, which may be linear or cyclic, as described in DE 198 60 361 and PCT/FR99/02345 both as monomers and with their corresponding starter systems for photocuring, hot-curing or cold-curing mixtures.

Hydroxyl-containing materials can be added; the addition of vinyl ether compounds to the epoxy resin can be useful.

Further ring-opening monomers can be compounds of orthocarbonic acid, reference being made here by way of example to the basic structure of spiro-orthocarbonic acid (Beyerlei et al. U.S. Pat. No. 5,556,896):

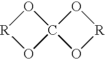

Ormocer matrices, as described in WO 92/16571, DE 413 34 94 or DE 100 16 324, are also suitable.

Another group of curable monomers recently usable for restorative purposes includes silicone-based monomers.

For example, it is possible to use a resin matrix, as described in DE 39 155 92 A1 for a dental cement, which consists of a curing fluid, more specifically a silicone oil modified with carboxyl groups, and a curing accelerator, more specifically a metal oxide and/or a metal hydroxide.

For example, there may be suitable a resin matrix, as described in U.S. Pat. No. 3,127,363A, in which an organosiloxane (A) of the general formula XO—Si(R)₂—[O—SiR₂]ₙ—O—Si(R)₂—OX is subjected to cross-linking condensation with a trifunctional crosslinking agent (B), for example of formula RSi(OH)₃.

A two-component resin matrix may also be suitable in which one of the two components (component I) consists of or comprises one or more silicone oils having at least two SiH groups, and the other of the two components (component II) consists of or comprises one or more silicone oils having at least two vinyl groups, and one of the two components additionally contains a catalyst for an addition reaction or addition crosslinking of the two components I and II.

With such mixtures it is possible to prepare, for example, root canal filling mixtures, such as are described in EP 0 864 312 B1 and which, for example by addition of nano alkaline earth fluorides, are able to effect therein transport of fluorine into extremely fine dentine canaliculi.

Another significant group in this connection relates to materials the curable matrix of which is based on cement systems (curing reaction of acid group components with basic components). Classic cements in the field of dentistry and medicine consist primarily of acids and reactive alkaline glasses or metal oxides. The term “classic” cements denotes resin-free cements. Resin-modified versions of those cements have easily been prepared by mixing with polymerisable resins. Cements contain a number of characteristic groups:

The classic dental polyalkenoate cements contain finely ground fluoroaluminium silicate glass powder (or basic oxides), polyalkenoate acids and water. The formation of water-insoluble dental polyalkenoate cements from the water-based formulations is effected in several steps and over a prolonged period. In summary, the polyalkenoate acid attacks the fluoroaluminium silicate powder in the presence of water at acid-accessible locations and as a result ions (specifically alkaline earth metal ions and aluminium ions) are released. Those released metal ions enter into an ionic bond with alkenoate groups of the polyalkenoate acids and form a crosslinked structure. In that way, the salt-like cement becomes a water-insoluble hard material. The curing or setting of the polyalkenoate cement to form that water-insoluble material takes place very quickly in the first few minutes. As setting progresses, the reaction slows down over several hours to several days. During that time, the cement is not yet resistant to saliva.

Suitable crosslinkable acids of polyalkenoate acids are, for example, polyacrylic acid, polyitaconic acid, polymaleic acid, lactic acid, polyvinylsulfonic acid, polystyrenesulfonic acid, polysulfurous acid, polyvinylphosphonic acid and polyvinyl-phosphoric acid or copolymers thereof. The main constituents of polyalkenoate cements can contain, for example, fluoroaluminium silicate glass powders. They consist of from 10 to 25% by weight Al, from 5 to 30% by weight Si, from 1 to 30% by weight F, from 0 to 20% by weight Sr, from 0 to 20% by weight Ca and from 0 to 10% by weight alkali metal ions (Na⁺, K⁺, etc.), based on the total weight of the glass. They are prepared by mixing together the individual components and melting the mixture. The material is then cooled and ground to an average particle size of from 0.2 to 20 μm.

When basic oxides are used for the preparation of the water-based polyalkenoate cements, it is preferable to use zinc oxide or zinc oxide and deactivated zinc oxide/magnesium oxide. Also preferred are the more recent zinc-based dental mixtures from EP No. EP 0 883 586. Other basic oxide cements contain such oxides of Be, Cu, Mg, Ca, Sr, Ba and combinations thereof.

The “classic” dental silicate cements contain finely ground fluoroaluminium silicate glass powder (or basic oxides), phosphoric acid and water.

The “classic” dental phosphate cements contain finely ground modified zinc oxide powder (or other basic oxides), phosphoric acid and water.

Other classic dental cements can also be cements that are composed mainly of zinc oxide or other metal oxides and of weak organic acids or phenol-containing compounds (e.g. eugenol).

The dental cements can have a powdery, fluid or pasty consistency. In addition to the composition of the dental cements in accordance with the present invention it is possible to add UV-light-absorbing substances, plasticisers, anti-oxidants, bactericides, surfactants etc.

Important constituents of the above-mentioned curing materials or cement systems are nanostructured alkaline earth fluorides MF₂

wherein M denotes the alkaline earth cation Ca²⁺, Mg²⁺, Sr²⁺ or Ba²⁺ and F denotes the fluoride anion. The particle size of the alkaline earth fluoride is predominantly in the range of from 2 to 200 nm.

Calcium fluoride is especially important. Calcium and fluoride are essential constituents of the apatite of the hard tooth structure (Ca₁₀(PO₄)₆(OH, F)₂.

The presence of strontium in the apatite of the tooth is regarded as important for use in dentistry in connection with a possible caries-inhibiting effect (in addition to the suspected effect of reducing dentine sensitivity) and it imparts to the apatite a reduced solubility and a higher resistance to thermal influences. In addition, X-ray opacity is increased.

Nanocrystalline materials are generally synthetically produced materials which are characterised by continuous phases or by granular structures and a length of usually less than 200 nm. In dependence upon the number of dimensions in which those materials have a nanostructure, a distinction is made between (i) zero-dimensional, (ii) one-dimensional, (iii) two-dimensional and (iv) three-dimensional materials (R. W. Siegel, in Materials Science in Technology, Vol. 15: Processing of Metals and Alloys, R. W. Chan, 583 (1991)).

The specific properties of the nanocrystalline materials result from three basic features, namely (i) the atomic size range of ≦200 nm, (ii) the high proportion of atoms participating in the interfaces and (iii) the interactions between the individual sub-regions.

In the case of particle sizes in the nano range there is a high proportion of surface molecules in relation to the total number of molecules of a particle. In a material having an average particle size of 10-15 nm, from 15 to 50% of the atoms participate in the “particle interfaces”.

Because the number of interfaces in nanocrystalline materials is very much higher than in conventional materials, by suitable control in the course of the synthesis of the materials the nature of the interactions between the interfaces of all phases involved can be affected by the nature of the interfaces.

The interaction between nano alkaline earth fluoride in a polymeric matrix and its biological environment is very much more intensive than the interaction obtained in the case of alkaline earth fluorides on a μm scale in that matrix. As this invention shows, materials based on nano alkaline earth fluorides have consequently proved to be very useful dental restoration materials.

A further important property of the nano-dimensions of the nano alkaline earth fluorides is that refraction of light in the visible range, that is to say light of wavelength 400-900 nm, no longer occurs. The nanoparticles are too small to interact with the light which has a significantly longer wavelength.

As a result, in the case of nanoparticle sizes even fillers such as alkaline earth fluorides having refractive indices of from 1.37 to 1.47 in customary polymerised resin matrices having refractive indices of from 1.49 to 1.54, which would otherwise result in white-opaque mixtures and white-opaque cured solids, result in highly transparent mixtures suitable for dental aesthetic applications.

The nanoparticle size of the alkaline earth fluorides to be used in the dental material according to the invention is predominantly in the range of from 2 to 200 nm, preferably from 2 to 90 nm.

In a preferred form of the invention, the nano alkaline earth fluoride particles are surface-treated in order to obtain better incorporability into the resin matrix as well as better mechanical properties of the polymerised products.

For example, alkaline earth fluoride fillers can be surface-treated with mono- or poly-functional methacrylate esters of phosphoric, phosphonic or carboxylic acids or combinations thereof. Special preference is given to the esters of hydroxylethyl methacrylate and glycerol dimethacrylate. Poly-methacrylated polyvinylphosphoric acids or polymethacrylated polyvinylphosphonic acids or polymethacrylated polycarboxylic acids may also be suitable for surface modification. Further advantageous reactants for a surface treatment can be corresponding phosphoric acid esters, phosphonic acid esters or carboxylic acid esters having vinyl groups. Maleic acid can likewise be helpful. The choice of the surface pre-reactant depends upon the reactive groups of the resin matrix chosen, for example methacrylates for methacrylate resins, vinyl ether groups or epoxides for epoxy resins, vinyl or SiH groups for addition-crosslinking vinylsiloxane matrices.

It should accordingly be made possible to effect an organic modification of the particles at the surface that is advantageous for the particular intended application, which modification should then, by virtue of covalent bonds, provide higher mechanical strength.

The choice of the surface treatment agent can also be made, however, so that only the dispersivity of the nano alkaline earth fluoride and the degree of filling therewith is increased, without resulting in a mechanical reinforcement. Examples of that kind are treatment with phosphate salts or with non-organosilanes.

A further method is the application of a SiO₂ or ZrO₂ layer likewise on a nanometre scale and subsequent treatment with a reactive-functional silane such as, for example, methacrylic, glycidyl, amino or vinyl organosilanes, provided that the properties of the ion exchange are not too greatly affected thereby.

The nano alkaline earth fluoride fillers can also be incorporated into one of the above-described resin matrices, polymerised and ground and then used as filler, both with and without the surface treatment of the kind described above.

According to the invention, the nano alkaline earth fluoride fillers are contained in the dental material in an amount which, on the one hand, is sufficient to allow ion exchange with the biological environment and, on the other hand, which ensures that good mechanical properties are preserved. It is therefore advantageous for the proportion by weight to be from 1 to 70% by weight, especially from 1 to 30%, based on the restoration material. For a high mechanical strength, a modification in which the proportion by weight is from 1 to 15% by weight, based on the total weight of the dental material, is especially advantageous.

In addition to the essential and characteristic filler component nano alkaline earth fluoride, the dental material may also contain further fillers as optional but nevertheless very advantageous and no less preferred components.

Further fillers can be ground powders of SiO₂ and/or glass ceramics and/or glass, and/or microbeads of an average particle size of from 0.2 to 40μ, inorganic and/or organic fibres, other nano fillers such as SiO₂ and metal oxides of nanoparticle size, agglomerated nano fillers, unfilled or filled chip polymers, ion-releasing glasses or reaction products thereof or other fillers having active-ingredient-releasing properties or for increasing X-ray opacity.

As ground powders there are preferably used Ba, Sr or Li Al silicate glasses having an average particle size in the range of from 0.2 to 2.0 μm and refractive indices of from 1.49 to 1.53.

Preferred microbeads are fillers as described, for example, in DE-A 32 47 800. The average primary particle size is in the range of from 0.1 to 1.0 μm, especially from 0.15 to 0.5 μm.

The inorganic or organic fibres can be glass, aluminium oxide, polyethylene oxide or carbon fibres and can serve for oriented reinforcement.

Other nanofillers or agglomerated nanofillers based on SiO₂, optionally coated with metal oxides, or based on metal oxide are polydisperse or monodisperse and can also be in the form of a mixture of polydisperse and monodisperse spheres. They are described, for example, in W. Stöber et al. in J. Colloid and Interface Science 26, 62 (1968) and 30, 568 (1969), U.S. Pat. No. 3,634,588, EP 0 216 278, EP 0 275 688.

Sintered fillers, as described, for example, in EP 0 113 926, also come into consideration as an additive.

If desired, it is also possible for further fillers to be used to obtain increased X-ray opacity, but the average primary particle size of such fillers should not exceed 5.0 μm. Such fillers are described, for example, in DE-OS 35 02 594.

For the purpose of better incorporability or for better mechanical properties, the fillers can likewise be surface-treated in accordance with methods similar to those mentioned for the nano alkaline earth fluorides.

Alternatively, for that purpose it is also possible for nano-fillers according to the invention and/or other nanofillers or agglomerated nanofillers or other extremely finely particulate fillers to be polymerised in a hard matrix and then finely ground prior to the actual incorporation into the dental material. If desired, small amounts of optionally silanised microfine, pyrogenic or wet-precipitated silica can be incorporated into the dental material to adjust the viscosity, but in a maximum of 50% by weight, based on the dental material. Preference is given to from 1 to 25% by weight, especially from 3 to 10% by weight.

It is also possible to add other bioactive or antibiotic substances, as described, for example, in PCT/US 96/17871, as further constituents.

By virtue of their excellent optical properties, the restoration materials described according to the invention can be used in the visible region of the oral cavity and therefore in direct contact with saliva.

The alkaline earth fluoride component of the restorative material can therefore yield ions (inter alia fluoride, calcium) into the saliva and the surrounding enamel. The effect sought inter alia according to the invention, namely the exchange of ions with the biological environment of the dental material combined simultaneously with a high degree of transparency and hardness of the bioactive material, can be achieved using nano alkaline earth fluorides as filler in polymerisable monomers and in cements.

The filler according to the invention can thus be used in typical dental formulations such as lightly filled resins, composites, ormocers, giomers, compomers, resin-reinforced cements, classic cements (for example classic polyalkenoate cements, silicate cements, phosphate cements), oxiranes, siloranes or silicones, which are employed as tooth fillings, inlays or onlays, fixing cements, veneer materials for crowns and bridges, materials for artificial teeth, dentine and enamel bondings, lining materials, stump build-up materials, root-filling materials or other curable materials for prosthetic, conservative or preventive dentistry.

The invention is described extensively below with reference to exemplary embodiments and comparison examples.

### Example 1

### Preparation of Nanocrystalline Calcium Fluoride

Nanocrystalline calcium fluoride was crystallised from a ternary microemulsion. For that purpose, an aqueous phase containing KF (Merck, Darmstadt, Germany) was emulsified into a mixture of Brij 35® (polyethylene glycol lauryl ether, J. T. Baker, Deventer/Holland) and Octan (Sigma-Aldrich, Schnelldorf, Germany) in a fixed weight ratio of 3:7. The microemulsion was stirred vigorously at 30° C. with 50% by weight 1.5 mol KF to give a microemulsion. Again with vigorous stirring, a stoichiometric amount of an aqueous solution containing 0.75 mol CaCl₂.6H₂O (Riedel de Häen) was slowly added and the mixture was left to stand for 24 hours at 30° C. The residue was separated by centrifugation, dispersed twice with alcohol and once with water in an ultrasound bath (Bandelin Sonorex) with the aid of an Ultra Turrax® T 25 basic at 24,000 rev/min for 5 min, then washed and in each case separated by centrifugation. The moist calcium fluoride was then ultrasound-treated for 4 hours and immediately afterwards freeze-dried for 96 hours (water content<800 ppm).

The ultrafine powder was tested for crystallinity, particle size and identity.

X-ray diffractometer diagrams, in addition to showing a high degree of crystallinity, show identical refraction patterns to a corresponding calcium fluoride reference.

The particle sizes determined using a particle sizer (Horiba La. 920, Retsch Technologie, Germany) were 110-150 nm.

The recorded IR spectra of the nano calcium fluoride synthesised in Example 1 agree with a comparison substance (CaF₂ p.a., Merck, Darmstadt).

### Example 2

A composite having a content of 12% nano calcium fluoride was prepared. For that purpose, the various amounts of fillers were added to a resin matrix based on photocurable dimethacrylates. By photocuring in a photocuring apparatus Dentacolor XS (Kulzer, Germany) the resulting pastes were used to produce test specimens which were tested for different properties.

The resin matrix used was prepared from
10 parts triethylene glycol dimethacrylate
10 parts Bis-GMA
10 parts urethane dimethacrylate
0.05 part camphorquinone
0.05 part dimethylaminoethyl methacrylate
2 parts Aerosil 202 and the following fillers were incorporated into 20 parts of the resin matrix:

### C MH (Comparison Example)

78 parts barium aluminium borosilicate glass 0.7 μm, methacryl-silanised
0 part nano calcium fluoride according to Example 1
   C MH/CaF (according to the invention)
66 parts barium aluminium borosilicate glass 0.7 μm, methacryl-silanised
12 parts nano calcium fluoride according to Example 1

To evaluate the physical and fluoride-release properties of this nano-calcium-fluoride-filled composite, in addition to the comparison sample there were additionally used a resin-modified cement (LC Vitrofil, Dental Fillings, Brazil) and a compomer (Ionomolar, Dr. Ihde, Germany) and shown in Table 1.

TABLE 1

| | | Composite | | |
|---|---|---|---|---|
| | | C MH/CaF | Resin- | |
| | Composite | (according to | modified | |
| Test procedure | C MH | the invention) | cement | Compomer |
| Cure depth | 6.1 | 6.0 | 7.3 | 6.8 |
| [mm], 30 sec. | | | | |
| Opacity [%] | 81 | 82 | 83 | 86 |
| Bending strength | 162 | 168 | 73 | 134 |
| [MPa] | | | | |
| Elasticity | 10.6 | 14.4 | 9.85 | 14.4 |
| modulus [GPa] | | | | |
| Barcol hardness | 80 | 79 | 70 | 81 |
| Water absorption | 22.5 | 72 | 92 | 28.3 |
| [μg/mm³] | | | | |
| Water-solubility | 1.9 | 11.8 | 10.5 | 3.1 |
| [μg/mm³] | | | | |

Table 1 shows that
1. the cure depth is good and comparable to the composite material C MH, resin-modified cement and compomer material,
2. the opacity is good for the purpose of producing aesthetic restoration materials and is comparable to the other tooth-filling materials mentioned,
3. the bending strength and elasticity modulus are comparable to those of the load-bearing composite,
4. the Barcol hardness corresponds to the hardness values of the other filling materials.
5. the water-solubility is very slightly higher, but there is a greater release of fluoride with high physical strength in comparison with composites (see below).

The important property of the bioactive release of ions was demonstrated by reference to the high level of fluoride release of the C MH/CaF, in comparison with the CaF₂-free comparison composite C MH and the compomer and the “resin-reinforced cement” which, as is known, exhibits significant fluoride release.

For that purpose, 4 test specimens having a diameter of 2 cm and a thickness of 2 mm with a total surface area of 45 cm² were placed in 100 ml of water and the fluoride release was measured.

Measurements were carried out in the range of from 0 to 90 days.

The results are shown in Diagram 1.

It can be seen from the diagram of fluoride release that
1. the order of magnitude of the fluoride release rate is or can be of a similar order of magnitude to the fluoride release of the “re-inforced cements”,
2. the order of magnitude of the fluoride release rate is greater than in the case of the compomer tested, which is a photocurable single-component glass ionomer cement in paste form,
3. the composite formulation without nano calcium fluoride (C MH), which represents the state of the art of composites, exhibits almost no or extremely low fluoride release or fluoride release rate.

### Example 3

As an example of use of nanocrystalline calcium fluoride in a photocurable self-etching veneer cement, the following paste was prepared:
47.5 parts Artegral One (Merz Dental, Germany) (self-etching single-component adhesive)
40.7 parts barium aluminium borosilicate glass 0.682 , meth-acrylsilanised
11.8 parts nanocrystalline calcium fluoride from Example 1

The above components were used to prepare a paste and, for testing different properties and for the adhesion test, suitable test specimens were produced from the resulting paste with the aid of a halogen lamp (Translux EC from Kulzer, Germany).

The results of the measurements of the physical properties on photopolymerised test specimens are shown in Table 2:

TABLE 2

| | Photocurable self- |
|---|---|
| | etching veneer cement |
| Cure depth 40 sec | 6.60 |
| irradiation [mm] | |
| Opacity [%] | 75.9 |
| Bending strength [MPa] | 147.5 |
| Elasticity modulus [GPa] | 5.568 |
| Barcol hardness after | 70/70/71 |
| 10 min | |
| Self-adhesive force of the | 18.0 |
| composite veneer on enamel | |
| N/mm² | |

The above-listed formulation is excellently suitable as a photocuring self-etching adhesive for composite or methacryl-silanised ceramic facets on enamel.

The adhesive cement is also excellently suitable for attaching decorative jewellery (twinkles) or orthodontic brackets to teeth in a way that is easy on the teeth, because its fluoride-release capacity is able especially to effect fluoride-hardening of the hygiene-compromised zone around those objects.

### Example 4

As an example of use of nanocrystalline calcium fluoride in ring-opening monomer mixtures, the following highly fluid dry-hardening tooth varnish was produced:
75.6 parts Silbione UV Polymer 30 (Rhodia, France)
4.1 parts Rhodosil Photosensitizer (Rhodia)
11.2 parts Silbione UV PI+(Rhodia)
9.1 parts calcium fluoride from Example 1

The resulting calcium-fluoride-filled varnish can be applied to the tooth in a very thin and almost invisibly transparent way and cured with the aid of a halogen lamp (Translux EC from Kulzer, Germany). The cured varnish without a smear layer protects the tooth mechanically and by means of fluoride-hardening.

For testing the opacity, 2 mm thick test specimens were produced from the resulting highly fluid varnish paste.

In comparison with the unfilled resin system, the resin matrix filled with nanocrystalline calcium fluoride exhibits an astonishing low change in opacity (the opacity of filling materials is 60-90%).

Opacity of the unfilled resin: 19.6%.

Opacity of the resin filled with 9.1% calcium fluoride: 43.8%.

### Example 5

A further example of use of nanocrystalline calcium fluoride is the preparation of a filled self-etching adhesive for restorations with photocurable and self-curing composite and compomer filling materials. For that purpose, the following modified adhesive was prepared:
95 parts “Autobond” part A (Apol, France)
5 parts nanocrystalline calcium fluoride from Example 1

The resulting modified self-etching adhesive lightly filled with nano calcium fluoride was mixed with the unmodified part B of the “Autobond” and applied to the tooth with photocurable composite “Apol” in accordance with the manufacturer's information on use.

The adhesion values of 16.29 MPa on enamel and 12.57 MPa on dentine are of the order of magnitude of the unmodified Autobond system. Unlike the Autobond system, however, the new calcium-fluoride-filled adhesive has the ability to yield fluoride ions and thus to strengthen the treated hard tooth structure.

1-22. (canceled)

23. A curable restoration material for teeth comprising (i) at least one crosslinkable resin system and/or at least one curable acid/base cement system, and (ii) at least one nanostructured alkaline earth fluoride, the particle size of the alkaline earth fluoride being predominantly in the range of from 2 to 200 nm.

24. A curable restoration material of claim 23 wherein the material comprises a crosslinkable resin system comprising monomers that are free radically polymerisable.

25. A curable restoration material of claim 24 wherein the monomers are (meth)acrylates.

26. A curable restoration material of claim 23 wherein the material comprises a crosslinkable resin system comprising monomers that are polymerisable with ring-opening.

27. A curable restoration material of claim 26 wherein the monomers are epoxides.

28. A curable restoration material of claim 23 wherein the material comprises a crosslinkable resin system comprising monomers that are crosslinkable silicones.

29. A curable restoration material of claim 28 wherein the monomers are addition-crosslinking vinyl silicones.

30. A curable restoration material of claim 23 wherein material comprises a curable acid/base cement system that comprises a polyalkenoate cement.

31. A curable restoration material of claim 23 wherein the material comprises a curable acid/base cement system that comprises a silicate cement.

32. A curable restoration material of claim 23 wherein the material comprises a curable acid/base cement system comprises a phosphate cement.

33. A curable restoration material of claim 23 wherein the material comprises a curable acid/base cement system that comprises zinc oxide and/or other metal oxides and/or weak organic acids and/or phenol derivatives.

34. A curable restoration material of claim 23 wherein alkaline earth fluoride comprises calcium fluoride.

35. A curable restoration material of claim 23 wherein the alkaline earth fluoride comprises strontium fluoride.

36. A curable restoration material of claim 23 wherein the alkaline earth fluoride is surface-treated.

37. A curable restoration material of claim 36 wherein the alkaline earth fluoride is surface-treated with esters of phosphoric acids, phosphonic acids or with esters of carboxylic acids.

38. A curable restoration material of claim 23 wherein the curable restoration material comprises from 1 to 70% by weight alkaline earth fluoride.

39. A curable restoration material of claim 23 wherein the curable restoration material comprises from 1 to 30% by weight alkaline earth fluoride.

40. A curable restoration material of claim 23 wherein the curable restoration material comprises from 1 to 15% by weight alkaline earth fluoride.

41. A curable restoration material of claim 23 wherein the material comprises fillers of SiO₂ and/or glass ceramics, and/or glasses and/or microbeads and/or inorganic fibres and/or organic fibres.

42. A curable restoration material of claim 23 wherein the total filler content of alkaline earth fluoride and additional fillers is from 1 to 95% by weight.

43. A process for preparation of a curable restoration material of claim 23, comprising:
curing at least one crosslinkable resin system and/or at least one curable acid/base cement system which comprising alkaline earth fluorides and optionally additional fillers is cured, finely ground to a particle size of from 0.2 μm to 100 μm and then used again as filler.

44. A method of restoring teeth comprising using a curable restoration material of claim 23.

45. A method of claim 44 wherein the material is applied to a tooth of a subject.
